(19) United States
(12) Patent Application Publication
(10) Pub. No.: US 2008/0152733 A1
(43) Pub. Date: Jun. 26, 2008

(54) Noni juice composition

(76) Inventor: Lawrence M. Logsdon, Laguna Niguel, CA (US)

Correspondence Address: Lawrence M. Logsdon;Tropical Functional Labs, 7111 Arlington Avenue, Suite F, Riverside, CA, 92503, US

(21) Appl. No.: 11/642,279

(22) Filed: Dec. 20, 2006

(51) Int. Cl. A61K 36/45; A61K 36/00; A61K 36/73; A61K 36/87

(52) U.S. Cl. 424/732; 424/765; 424/766; 424/769

(57) ABSTRACT

A novel noni therapeutic juice composition is provided comprising juice of noni fruit possessing substantially improved potency and stability; a blueberry composition; and a coenzyme precursor to tryptophane. A superior and biologically potent noni results which is synergistically enhanced with a blueberry juice composition and a coenzyme precursor to tryptophane.

### I. FIELD OF THE INVENTION

The present invention is directed to therapeutic compositions for human consumption comprising powerful antioxidant formulas containing the juice of the noni fruit, known as morinda citrifolia.

### II. BACKGROUND OF THE INVENTION

For over 2,000 years noni (morinda citrifolia) has been used for its beneficial properties; for example, Polynesians used noni as a general tonic that they found particularly beneficial for imbalances of the immune, respiratory, digestive and intestinal systems. Noni also appeared to its early users to aid the central nervous system, injured muscles, bones and tissue. The usage of noni's beneficial and therapeutic properties was passed from parents to children for countless generations in the South Pacific and Southeast Asia. There the noni juice was used both internally and externally for its innumerable benefits.

Morinda citrifolia, alternatively known as Indian mulberry and most commonly known today by the same name ancient Hawaii healers gave it as simply noni, was historically employed to treat conditions that affected the respiratory, digestive, nervous and immune systems. In addition, it was also employed historically to treat joint, skin and skeletal problems. The earlier usage of noni by traditional healers for medicinal use employed every part of the noni plant: leaves, roots, bark, seeds, flowers and fruit. The noni leaves were found to be particularly useful for external inflammations as well as wounds and pain relief; whereas root extracts were found to lower blood pressure; the bark was employed for its strong astringent properties and also to treat malaria; the seeds exhibited a purgative action; and the flower extracts were found to relieve eye inflammations. It was early recognized that the noni fruit was the most prized part of the plant, having numerous applications including many of those recited above.

In some ancient cultures, noni was found to be nutritious and was employed as a dietary staple and in other cultures consumed as a source for nourishment in times of famine. The oldest references relating to noni date back tens of centuries to India's ancient sandscript writings that cite the use of och plant in ayurvedic medicine. Historians believe that noni originated in Southeast Asia and during an ancient migration to colonize new lands, it was brought to Micronesia and Polynesia. The latter has a rich healing heritage recorded, and knowledge about noni's medicinal uses was handed down from generation to generation. Scientific literature regarding noni is far more substantial and abundant from this region of the world than from India or any other region.

The typical noni plant is an evergreen that can range from a small bush to a tree 30 feet tall. The noni bush produces a lovely fragrant white flower that blooms year-round, containing noni fruit which has a lumpy texture, very high in fiber, and appears similar to a medium-sized potato but, however, has a rancid taste and smell when ripened.

The noni fruit is covered with reddish-brown pits that contain seeds which typically have an attached air sac allowing the seeds to float for a month in the ocean. This is historically believed to be the mechanism that transported noni to various countries centuries ago.

Polynesian healers and historians insist that the noni plant was considered sacred and brought intentionally to the islands primarily because of its medicinal properties. Additionally, it was valued for its nutritional properties as well as its use as a dye.

Upon colonizing new lands, ancient ocean-going Polynesians made voyages from island to island in their canoes, bringing only the essentials to survive: food, clothing, tools and medicinal plants which included their sacred noni plant.

Traditionally, the noni plant was employed by early Polynesians to treat pain, inflammation, burns and other afflictions, intestinal worms, nausea, food poisoning, fever, infections, wounds, diarrhea, constipation, menstrual cramps, insect and animal bites, and conditions of aging. Notwithstanding these beneficial effects, it was difficult for early Polynesians to ingest the noni juice because of its rancid smell and taste. This was found to be especially true in the early and mid-1900s when noni was very unpopular in many Polynesian communities because of its unpleasant smell and many trees were cut down to clear residential areas of the noni's foul odor. Prior to the 1900s, Christian missionaries who came to Polynesia banned Huna, the term for Polynesian natural healing, and medicinal interest in noni somewhat diminished. In recent times, the advent of (allopathic) synthetic drugs began to diminish the interest in the amazing noni until now. For thousands of years people have used food, herbs, fasting, colon cleansing, and other natural methods to strengthen the immune system and to prevent and cure disease. According to the World Health Organization figures, herbal medicine is still the most widely practiced form of medicine in the world today.

Despite the advent of synthetic drugs and the concurrent diminishing interest in noni, upon experiencing the often serious and substantial side effects that accompany synthetic drugs, which were found to treat the symptoms rather than the cause of the afflictions, a renewed interest emerged in therapeutic natural compositions such as noni.

The allopathic medical approach to treating infectious diseases, which was relatively new at the time, began to lose favor because of changing pathogenic microbes. Antibiotic resistant diseases began to reach epidemic proportions in the world and the effectiveness of the antibiotic is rapidly decreasing, causing a greater interest by scientists in studying botanicals that traditional healers have proven effective for fighting disease, with noni emerging as an extremely valuable botanical medicine.

In today's alternative medicine world, noni fruit enjoys a highly-prized position as a medicinal commodity, just as it was in ancient times. However, modern methods of extracting and preparing noni juice to eliminate much of the unpleasant smell and taste without compromising beneficial properties continue to require further research and development. Additionally, noni is found to produce therapeutic results in aiding health problems such as immune system disorders, bacterial and viral infections, respiratory problems, allergies, arthritis, diabetes, cancer, high blood pressure, menstrual cramps, poor digestion, tuberculosis, malaria, influenza, drug and alcohol addiction, among other afflictions.

The vast therapeutic spectrum of treatments attributable to noni is dramatically witnessed by testimonials from users who have experienced dramatic results. Numerous doctors have documented that countless patients have been assisted by the noni juice. Scientific studies have shown that noni has the potential to stimulate the immune system, inhibit tumor growth, regulate proper cell function, purify the blood, and regenerate damaged cells, among other properties. These broad-ranging results have inspired researchers to continue to explore and develop better methods of producing, harvesting, transporting, purifying and blending this fruit and compositions which optimize its therapeutic benefits.

Modern day drugs used to treat illnesses and diseases are generally referred to as pharmaceuticals. Phytochemicals in plants having potential health benefits are referred to an nutraceuticals, of which noni is found to contain a variety of beneficial phytochemicals, some of which are not found in any other known plant. These phytochemicals work synergistically to enhance biologically the expression of each individual component found in noni so that scientists are incentivized to optimize these therapeutic qualities of noni to dramatically improve its disease preventing and health promoting benefits.

Noni (morinda citrifolia) is said contain over 150 nutraceuticals, among which are the following: Xeronine, Scopoletin, Proxeronine, Morindadiol, Proxeronase, Rubiadin, Serotonin, Magnesium, Damnacanthal, Carbonate Nordamnacanthal, Protein, Antraquinones, Sodium, Carotenoids, Bioflavinoids, Morindine, Morindone, Terpenes, Plant Sterols, Iron, Sitosterol, Phosphate, Glycosides, Carbohydrates, Aleizarin, Acetin, GlucoP, Ursolic acid, Caproic acid, Caprlyic, Glucopyranose, Asperuloside, Precursors, Vitamins, Trace elements, Alkaloids, Enzymes, Serine, Multi-receptor activators, Chlororubin, Methionine, Alanine, Isolucine, Arginine, Leucine, Aspartate, Lysine, Cysteine, Phenlyalanine, Cystine, Threonine, Glycine, Trypophane Glutamate, Valine, Tyrosine, Histadine, and Proline.

Among these noni possesses a rich source of Proxeronine, Proxeronase and Xeronine, which are alkaloids employed by the body to strengthen and revitalize cells, as well as possessing a rich source of antioxidants and essential nutrients.

Consequently, substantial research has been performed worldwide by modern scientists that have led them to acknowledge noni's unique healing properties through its high quantities of phytonutrients and enzymes. Beginning in the 1950s, noni has been researched at leading institutions in France, the Netherlands, Germany, Ireland, Taiwan, Japan, Austria, Canada and Hawaii.

In 1993 a Japanese research team reported in the Cancer Letters that noni was the most effective of 500 tropical plant extracts at preventing the growth of tumors. Studies have shown that noni stimulates the production of T-cells in the immune system. Other studies have shown noni to have very effective anti-inflammatory analgesic antibacterial properties. One study at the University of Hawaii isolated one of the 140 compounds, scopoletin, and demonstrated that it lowered high blood pressure, relaxed spasms, reduced inflammation, killed bacteria and fungi and prevented growth of tumors. Two reasons are postulated by scientists for noni's wide range of effectiveness are its ability to regulate cell function and cellular regeneration and its role as an adaptogen; i.e., helping the body to restore its organs to normal function or homeostasis.

Typical improvements when ingesting noni are illustrated in the following chart:

| | No. Taking | Percent | Primary |
|---|---|---|---|
| Condition Reported To | Noni for | Improve- | Active |
| Respond to Noni | Condition | ment | Component |
| Allergy, improved symptoms | 948 | 84% | Xeronine |
| Anti-Aging, look better | 148 | 78% | Terpene |
| Arthritis, lessened symptoms | 719 | 81% | Terpene |
| Breathing, improved | 2,854 | 77% | Xeronine |
| Cancer, reduced symptoms | 889 | 65% | Xeronine |
| Depression, lessened symptoms | 807 | 77% | Xeronine |
| Diabetes, Types I and II | 2,773 | 84% | Scopoletin |
| Digestion improved | 1,593 | 89% | Xeronine |
| Energy Increased | 8,327 | 92% | Xeronine |
| Fuzzy thinking, helped clear | 373 | 88% | Xeronine |
| Heart Disease, improved | 1,123 | 80% | Xeronine |
| symptoms | | | |
| High Blood Pressure, | 938 | 85% | Scopoletin |
| improved | | | |
| Kidney health, improved | 2,372 | 67% | Xeronine |
| Mental acuity, increased | 2,983 | 72% | Xeronine |
| alertness | | | |
| Muscle, increased body- | 816 | 71% | Protein |
| building | | | |
| Obesity, lost excess weight | 2,841 | 75% | Xeronine |
| Pain, including headaches, | 4,231 | 88% | Xeronine |
| improved | | | |
| Sexuality, enhanced enjoyment | 1,608 | 87% | Xeronine |
| Sleep, improved | 1,231 | 73% | Xeronine |
| Smoking, stopped | 452 | 58% | Xeronine |
| Stress, helped cope with | 4,113 | 72% | Xeronine |
| Stroke | 1,019 | 57% | Xeronine |
| Increased feeling of well being | 4,561 | 80% | Xeronine |

The literature is likewise rife with testimonials and anecdotal evidence of the effectiveness of the noni plant.

Thus there is seen to be a dramatic need for utilizing and optimizing the benefits of the noni plant by employing compositions to further enhance and synergize the therapeutic aspects of noni.

In U.S. Pat. No. 7,070,813, filed Oct. 31, 2002, issued Jul. 4, 2006, there is disclosed methods and formulations or compositions for the treatment of colon cancer and particularly for the inhibition, prevention and/or reduction of cancerous cell growth as well as the destruction of early stage cancerous cells within the colon region of a mammal, wherein the formulations and compositions comprise an identified amount or concentration of a processed morinda citrifolia product or an active ingredient therefrom as obtained from the Indian mulberry plant.

In U.S. Pat. No. 6,528,053, filed Feb. 12, 2002, issued Mar. 4, 2003, there is taught compositions and methods for reducing oxysterol buildup in the blood and normalizing cholesterol and blood pressure in a mammal employing compositions comprising morinda citrifolia extract, red wine extract, prune extract, blueberry extract, pomegranate extract, apple extract, and an enzyme mixture.

In U.S. Pat. No. 6,440,410, filed Nov. 28, 2001, issued Aug. 27, 2002, there are disclosed compositions and methods for reducing oxysterol buildup in the blood and normalizing cholesterol and blood pressure in a mammal employing compositions that comprise morinda citrifolia extract, red wine extract, prune extract, blueberry extract, pomegranate extract, apple extract, and an enzyme mixture.

In U.S. Pat. No. 6,436,449, filed Mar. 2, 2001, issued Aug. 20, 2002, there is disclosed the use of an extract derived from the fruits, leaves, the bark or the roots of morinda citrifolia L. for the manufacture of a medicament for the treatment of a mammal suffering from tinnitus. The extract may be a liquid present in the medicament in an amount such as to give a daily dosage of 0.1-2 ml, or 0.2-1 ml, e.g. 0.4-0.7 ml, per kg body weight of the patient. The extract also may be a solid present in the medicament in an amount such as to give a daily dosage of 5-200 mg, or 10-100 mg, e.g. 20-70 mg, per kg body weight of the patient. Optionally, the medicament also may comprise lycopene, vitamin C, coenzyme Q10 and an extract from the leaves of Ginkgo biloba. The medicament may be given e.g. by oral, rectal, transdermal or inhalation administration.

In U.S. Pat. No. 6,403,086, filed Nov. 30, 2001, issued Jun. 11, 2002, there are disclosed compositions and methods for reducing oxysterol buildup in the blood and normalizing cholesterol and blood pressure in a mammal employing compositions comprising morinda citrifolia extract, red wine extract, prune extract, blueberry extract, pomegranate extract, apple extract, and an enzyme mixture.

In U.S. Pat. No. 6,387,370, filed Jan. 19, 2001, issued May 14, 2002, there are disclosed compositions and methods for reducing oxysterol buildup in the blood and normalizing cholesterol and blood pressure in a mammal employing compositions comprising morinda citrifolia extract, red wine extract, prune extract, blueberry extract, pomegranate extract, apple extract, and an enzyme mixture.

In U.S. Pat. No. 5,288,491, filed Sep. 24, 1992, issued Feb. 22, 1994, there is disclosed a method for processing the noni plant into powder including the steps of picking the noni fruit from the tree, placing picked noni fruit in a room, washing and cleaning the noni plant, mashing the noni fruit, placing the pulp onto liner, rotating trays for five hours, rotating trays for another five hours, rotating trays for another 14 hours, and crushing and grinding dried wafers.

In U.S. Pat. No. 6,589,514, filed Apr. 17, 2001, issued Jul. 8, 2003, there is disclosed repair serums comprising morinda citrifolia from the Indian mulberry plant, providing significant skin care advantages not found in prior art intensive repair serums.

In U.S. Pat. No. 6,855,345, filed Nov. 1, 2002, issued Feb. 15, 2005, there is disclosed a unique natural formulation and method of administering the same to treat and prevent diabetes, or rather advances treatment of diabetes, by providing a naturaceutical composition or treatment formulated with one or more processed morinda citrifolia products as derived from the Indian mulberry plant. The morinda citrifolia is particularly adapted to treat Type II diabetes. The morinda citrifolia product is preferably a leaf extract, but may also be in the form of a juice, a puree juice, a dietary fiber, or other similar forms and is incorporated into various carriers or naturaceutical compositions suitable for in vivo treatment of a patient. The naturaceutical may also combine other food products into the naturaceutical, such as fruit juices, dietary supplements, vitamins and minerals, and others.

### III. OBJECTS OF THE INVENTION

It is therefore an object of this invention to provide an improved noni therapeutic composition that enhances the body's natural ability to fight infections, diseases, colds and flu.

A further object of this invention is to provide an excellent source of potent antioxidants in the form of polyphenyls to guard against damage from free radicals which cause cell damage, lead to premature aging, heart disease and other degenerative disorders.

Another object of this invention is to provide a noni therapeutic composition which acts as a detoxifying agent, cleansing the liver, neutralizing toxins and helping maintain a healthy digestive system.

Yet another object of this invention is to provide a noni therapeutic composition which comprises a substantial source of enzymes that assist improved digestion and absorption of nutrients, resulting in more energy and stamina.

Again another object of this invention is to provide a noni therapeutic composition that acts as a natural analgesic with anti-inflammatory properties that help manage pain and inflammation.

Again a further object of this invention is to provide a unique proprietary blend of noni, blueberries and raspberries containing powerful antioxidants that assist in anti-aging properties and help fight infections while inhibiting the spread of intestinal bacteria.

Yet again a further object of this invention is to provide phytochemicals which work synergistically to enhance biologically the expression of each individual component found in noni.

### IV. SUMMARY OF THE INVENTION

These and other objects of the instant invention are accomplished generally speaking by providing a therapeutic juice composition comprising the juice of noni fruit, a blueberry juice composition, and a coenzyme precursor to tryptophane.

The noni content of the instant invention is obtained from organically grown plants in rich volcanic soils growing wild without the use of pesticides, synthetic fertilizers and typically fed by rain water. These plants are picked at the peak of their maturity, typically transported in cold storage to facilities for extraction. A unique process, the subject matter of co-pending patent application U.S. Ser. No. ______ filed Dec. 13, 2006, entitled “Noni Juice Composition and Process Therefor”, rather than employing heat, utilizes infrared dehydration wherein the IR spectra is carefully adjusted to remove the water without in any way degrading or diminishing the potency of the resulting dehydrated fruit. This results in a most potent noni product which delivers at least 10,000 mg of noni in each ounce serving extracted from more than 60,000 mg of fresh noni fruit. The concentrated noni, after dehydration and resulting crystallization, is reconstituted by adding water and other innovative substituents. The noni content may alternatively be provided in a powder form, commercially known as Noni Powder Tahiti Trader, and in extract form known as Noni Extract F-118425.

A blueberry juice substituent with a maintained stable assay is added to the noni concentrate as above described. The blueberry juice substituent is produced in situ and normally comprises a blueberry extract in a concentrated juice form.

Another novel ingredient added to the noni concentrate blueberry mixture is a flavor based additive known commercially as 5 HTP which is a coenzyme precursor to tryptophane that assists in producing serotonin. This ingredient is found to provide catalytic action and result in serotonin production of 50 mg per 30 ml of the noni concentrate blueberry flavor based additive mixture.

Other ingredients may be added to the novel composition of the instant invention to add color, pleasant taste, appropriate consistency and fluidity, and further to synergize the composition of the instant invention. These ingredients would include filtered water to provide fluidity and consistency, xanthan gum, noni powder and noni extract to provide the noni content, grape white, pear puree, raspberry flavor, exberry grape blue stable and citric acid to result in a commercially acceptable and stable composition.

The above recited noni therapeutic juice composition is a powerful antioxidant formula which is highly bioactive, potent and is pleasing to the taste.

The resulting product is found to be far superior to other noni compositions by virtue of the synergistic action of the concentrated noni as produced, the additive of in situ blueberry juice, and the flavor additive as above described.

This composition is found to dramatically increase mental clarity and attention span, as well as allowing greater physical performance levels. In addition, it is found to significantly benefit the immune system by supporting the immune system's natural ability to fight disease and infection; the circulatory system tissues and cells by ridding the body of harmful free radicals and increasing energy levels; the digestive system by supporting proper digestion and assisting absorption of significantly more nutrients at the cellular level; and skin and hair receive therapeutic benefits from this composition.

In addition, this novel composition is found to alleviate depression and anxiety; improve pain management by naturally boosting serotonin and endorphin production; specifically supporting the immune system to help fight colds and flu and improve the healing process; assisting in the normalization of glucose and insulin levels; assisting in the normalization of blood pressure and the relief of hypertension; providing detoxifying action to cleanse the liver and neutralize toxins; and improve digestion and absorption of nutrients.

These as well as previous therapeutic benefits are obtained from the improved noni of the instant invention and all with no side effects which accompany the use of pharmaceuticals that treat the symptoms rather than the cause.

Although each substituent of the composition of the instant invention may be combined employing suitable percentages to maximize the varying benefits of these compositions, typical weight percentages of the substituents of the composition of the instant invention are as follows: water 65-80%, xanthan gum 0.05-1.5%, noni powder Tahiti Trader 0.25-0.75%, noni extract F-118425 (the commercial designation of the substituent) 3.5-7.5%, grape white 8-15%, pear puree 32 brix (the commercial designation of the substituent) 3-7%, blueberry WONF 65 brix (the commercial designation of the substituent) 1-2%, raspberry flavor FS 112870 (the commercial designation of the substituent) 0.3-0.7%, exberry grape blue stable (the commercial designation of the substituent) 0.1-0.15%, and citric acid 0.3-0.36%. Preferred of these are compositions with weight percentages as follows: filtered water at about 75%, xanthan gum at about 0.1%, noni powder Tahiti Trader at about 0.5%, noni extract at about 5.5%, grape white at about 11%, pear puree at about 5%, blueberry at about 1.5%, raspberry flavor at about 0.5%, exberry grape blue stable at about 0.13%, and citric acid at about 0.33%. Although the coenzyme precursor to tryptophane, bearing the commercial designation 5 HTP, may be present in any suitable form in any suitable substituent, typical compositions include the coenzyme precursor to tryptophane in the blueberry WONF 65 brix substituent. Any suitable weight percentage of the 5 HTP coenzyme precursor to tryptophane may be employed in the instant invention. However, it is found that the 5 HTP in a minimum weight percentage of at least about 0.1% of the total composition represents an effective amount to realize the benefits of the composition of the instant invention.

Any suitable xanthan gum may be employed in the composition of the instant invention. Typical xanthan gum substituents are those that are commercially available and well known to those skilled in the art.

Any suitable grape white substituent may be employed in the composition of the instant invention. Typical grape white substituents are those commercially available substituents well known to those skilled in the art.

Any suitable pear puree may be employed in the composition of the instant invention. Typical pear puree substituents are those that are commercially available and well known to those skilled in the art.

Any suitable raspberry may be employed in the composition of the instant invention. Typical raspberry substituents are those that are commercially available and well known to those skilled in the art.

Any suitable exberry grape blue stable may be employed in the composition of the instant invention. Typical exberry grape blue stable substituents are those that are commercially available and well known to those skilled in the art.

Any suitable citric acid may be employed in the composition of the instant invention. Typical citric acid substituents are those commercially available and well known to those skilled in the art.

While the present invention has been particularly described with respect to preferred concentrations of preferred substituents, it will be understood that the invention is not limited to these particular concentrations and substituents described in the preferred embodiments. For example, the optimal combination of this therapeutic composition may be varied accordingly by substituting for the above substituents, substitute substituents in suitable concentrations that possess the same or similar properties as outlined above to obtain the same or similar results as above recited. It is intended to cover all alternatives, modifications, and equivalents as may be included within the spirit and scope of the invention defined by the appended claims. Other ingredients may be added, for example, ingredients that provide further benefits specific to cardiac function as described in U.S. Pat. No. 6,932,989.

In addition, other substituents may be employed in the instant invention as claimed with similar results. In particular, the scope of the invention is intended to include, for example, adding in suitable concentrations the flavoring substituents to obtain a different or more pleasing taste for varying palates. In addition, other substituents may be added to enhance solubility and appearance of these compositions, in addition to other substituents which provide further synergy to these herbal compositions. For example, novel noni composition of the invention may be provided in any forms of delivery for human consumption other than liquid, such as capsules, concentrates, and the like.

1. A therapeutic juice composition comprising:
the juice of noni fruit
a blueberry juice composition; and
a coenzyme precursor to tryptophane.

2. The composition as defined in claim 1, wherein the juice of noni fruit is provided from a mixture of powdered noni fruit and extract of the noni fruit.

3. The composition as defined in claim 1, further comprising a puree of pear fruit.

4. The composition as defined in claim 1, further comprising white grape juice.

5. The composition as defined in claim 1, further comprising blueberry flavoring, xanthan gum, raspberry flavoring, citric acid, and water.

6. The therapeutic juice composition as defined in claim 1, wherein said juice of the noni fruit presents a stable assay and a potency that has not been degraded by dehydration.

7. The therapeutic juice composition as defined in claim 7, wherein said stable assay is obtained by dehydrating employing infrared wavelengths, satisfactory to remove water and not degrade fruit potency.

8. The therapeutic juice composition as defined in claim 7, wherein said stable assay is obtained by dehydrating employing infrared wavelengths, satisfactory to remove water and not degrade fruit potency.

9. The therapeutic juice composition as defined in claim 1, wherein said juice of noni fruit is present in a weight percentage of at least 30%.

10. The therapeutic juice composition as defined in claim 1, wherein said coenzyme precursor to tryptophane is present in a weight percentage that results in serotonin production of at least 50 mg per 30 ml of the noni fruit and blueberry juice composition.
